(19) United States
(12) Patent Application Publication
(10) Pub. No.: US 2008/0064100 A1
(43) Pub. Date: Mar. 13, 2008

(54) Method for Preparation of an Autologous Endometrial Culture for an Endometrium-Embryo Coculture

(75) Inventors: Dominique Vacher, Mougins (FR); Florence Cazals, Valbonne (FR); Christophe Soler, Nice (FR)

Correspondence Address: McLELAND PATENT LAW OFFICE, P.L.L.C., 11320 RANDOM HILLS ROAD, SUITE 250, FAIRFAX, VA, 22030, US

(73) Assignee: LABORATOIRES GENEVRIER SA, Antibes (FR)

(21) Appl. No.: 11/577,324

(22) PCT Filed: Oct. 14, 2005
(86) PCT No.: PCT/FR05/02555

§ 371 Date: Apr. 15, 2007

(30) Foreign Application Priority Data
Oct. 15, 2004 (FR) 0410931

(51) Int. Cl. C12N 5/08; C12N 5/06

(52) U.S. Cl. 435/373

(57) ABSTRACT

The invention relates to a method for the preparation, from an endometrial biopsy, of an autologous endometrial coculture system. The aforementioned method comprises steps yielding a first endometrial fraction referred to as the epithelial fraction, a second endometrial fraction referred to as the stromal fraction and a third endometrial fraction referred to as the mixed fraction which comprises a mixture of glandular and stromal cells, said method comprising in addition a step of purification of the aforementioned third fraction.

The present invention relates to the field of in vitro fertilization and specifically relates to a method for the preparation of an autologous endometrial coculture system for endometrium-embryo coculture.

In vitro fertilization (IVF) consists of reproducing in the laboratory that which occurs naturally in the fallopian tubes, namely fertilization and first stages of embryonic development. Since 1978, the year Louise Brown was born, this technique has made possible the birth of hundreds of thousands of children worldwide.

To evaluate the success rate of IVF techniques, several parameters are used in the literature. With respect to the number of procedures for the purpose of collecting oocytes, the following may be taken into account:
successful egg retrievals (greater than 95%);
embryo transfers (approximately 80%);
number of clinical pregnancies (pregnancy state defined by intrauterine sac and cardiac activity);
number of deliveries;
number of live births.

It is the number of deliveries with at least one normal live birth which probably best characterizes IVF technique success, this rate being approximately 12% to 15%. This rate heavily depends on the age of the patient, the number of treatment cycles and the number of embryos transferred.

Implantation failures following IVF can be of maternal or embryonic origin. One cause of implantation failure has been linked to the capacity of embryos to reach the blastocyst stage in culture, which in turn is related to culture conditions.

Traditionally, after in vitro fertilization, the embryo is placed in specific synthetic culture media chosen according to number of days in culture (day 1 to day 6). The embryo is then returned to the patient's uterus on day 2 or day 3, i.e., at the 4- or 8-cell stage after culture in a synthetic medium. The implantation rate of 2- or 3-day-old embryos remains low (approximately 15% per embryo). This low implantation rate can be explained in part by uterine hypermotility and by an inappropriate time of transfer: indeed, these embryos are placed in an inadequate environment because embryos at this stage of development in vivo are still in the fallopian tubes and the embryo reaches the uterus only at the blastocyst stage, i.e., five days after fertilization.

Coculture techniques (culture of an embryo up to the blastocyst stage on a layer of cells acting as a nutritional substrate) were first proposed in man in 1989 after many animal studies, the first in 1962, demonstrated that this technique improved the ratio of embryos reaching the blastocyst stage.

In addition to endometrium-embryo synchronization, the fact of being able to culture an embryo up to the blastocyst stage makes it possible to select embryos of high development capacity and to perform preimplantation diagnoses. In addition, embryo coculture techniques make it possible to obtain blastocysts of higher quality compared to blastocysts cultured in synthetic media.

Techniques for coculture of the embryo up to the blastocyst stage on animal cell lines, such as the VERO cell line from African green monkey kidney epithelium, for example, yield improved results compared to results from early day 2-3 transfers in synthetic media. However, these animal cells have not been validated for therapeutic use with respect to health safety according to existing legislation. The Decree of Jan. 12, 1999, establishing regulations regarding good MAP (medically assisted procreation) practices in France, specifies that only coculture on autologous cells does not lead to exogenous infection risks.

Out of an abundance of caution, coculture was abandoned by IVF laboratories in 1998-1999 to the benefit of synthetic media. New synthetic media then appeared which enabled culture of the embryo up to the blastocyst stage (so-called “sequential” media). However, none of these media could reproduce the coculture effect. Indeed, comparative studies between blastocysts arising from sequential media and those arising from coculture demonstrate that the blastulation rate is higher in coculture (approximately 45% compared to 30-35%), regardless of the coculture used, and that the blastocysts obtained are of higher quality (higher number of blastomeres, higher grade, lower fragmentation rate). The positive effect of coculture on embryonic development is related to embryo-cell interactions, to production of cytokines and/or embryotrophic and trophoblastic factors and to detoxification of the culture medium by somatic cells.

Consequently, the use of autologous somatic cell coculture appears as an ideal solution, one which will allow high implantation rates while preventing contamination of the embryo by exogenous viruses.

Among human somatic cells, endometrial cells are of particular interest. Indeed, they are the physiological cells of implantation and thus the ideal substrate for the embryo during coculture.

Autologous endometrial coculture systems, intended for use for endometrium-embryo coculture during IVF, consisting of a purified epithelial cell culture, are known. Their use remains limited, however, because glandular epithelial cell cultures are very delicate, have a limited lifespan of 2-4 days, only tolerate one culture cycle and always result in small quantities.

The applicant has noted, and it is one of the merits of the invention, that during the separation of the various cellular fractions from an endometrial biopsy, it is possible to obtain, in addition to a first endometrial fraction referred to as the epithelial fraction (primarily comprised of glandular cells) and a second endometrial fraction referred to as the stromal fraction (primarily comprised of stromal cells), a third endometrial fraction referred to as the mixed fraction which leads, after separating and purifying the glandular and stromal cells which comprise it and placing them in culture, to a mixed culture (of epithelial and stromal cells) of suitable quality for use in endometrium-embryo coculture during IVF.

The object of the present invention is to propose a method for the preparation of a novel autologous endometrial coculture system for endometrium-embryo coculture during IVF.

To this end, and according to a first aspect, the invention has as an object a method for the preparation of an autologous endometrial coculture system intended for use for endometrium-embryo coculture during IVF, the aforementioned culture system comprising epithelial cells and stromal cells, wherein the aforementioned method comprises steps yielding a first endometrial fraction referred to as the epithelial fraction, a second endometrial fraction referred to as the stromal fraction and a third endometrial fraction referred to as the mixed fraction which comprises a mixture of glandular and stromal cells and wherein said method comprises in addition a step of purification of the aforementioned third fraction.

In one embodiment, the step of purification of the aforementioned third fraction comprises: a) obtaining stromal cells from the third fraction, b) obtaining glandular cells from the third fraction and c) mixing the stromal and glandular cells thus obtained.

In one embodiment, the step a), obtaining stromal cells from the third endometrial fraction, referred to as the mixed fraction, comprises the operations of:
a1. placing in suspension the aforementioned third endometrial fraction in a suitable culture medium, thus yielding a first suspension;
a2. filtration of the aforementioned first suspension, thus yielding a first filtrate and a first fraction retained on the filter;
a3. centrifugation of the aforementioned first filtrate, thus yielding a first pellet containing stromal cells from the third endometrial fraction;
a4. taking up the aforementioned first pellet in an IVF medium, thus yielding a second suspension of stromal cells from the third endometrial fraction.

In one embodiment, step b), obtaining glandular cells from the third endometrial fraction, referred to as the mixed fraction, comprises filtration of the aforementioned first fraction retained and placing in suspension of the second fraction retained thus obtained, thus yielding a third suspension containing glandular cells from the third endometrial fraction, in an IVF medium.

In one embodiment, step c) consists of mixing the aforementioned second and third suspensions to yield a purified third endometrial fraction.

The mixture of glandular and stromal cells from the third endometrial fraction obtained in step c) is then placed in culture in an IVF medium. The cells are maintained in culture for several days.

In a preferred embodiment, the endometrial biopsy is cryopreserved immediately, in which case, approximately on the day the patient begins ovulating, the biopsy is thawed and treated according to the method described, and then the glandular and stromal cells from the purified third endometrial fraction are placed in culture.

In another embodiment, the endometrial biopsy is treated according to the method of the invention immediately after it is received by the laboratory. The mixture of glandular and stromal cells from the purified third endometrial fraction thus obtained is placed in culture for several days; said cellular culture is cryopreserved and thawed approximately on the day the patient begins ovulating.

In all cases, the method for the preparation of an autologous endometrial coculture system intended for use for endometrium-embryo coculture during IVF may comprise an additional step of packaging of the aforementioned autologous endometrial coculture system by means of a semisolid IVF medium, for use during transfer to a medically assisted procreation laboratory.

According to a second aspect, the invention relates to an autologous endometrial coculture system intended for use for endometrium-embryo coculture during IVF, the aforementioned system comprising a culture of epithelial and stromal cells from the purified third endometrial fraction.

According to a third aspect, the invention relates to the use of the autologous endometrial coculture system described for the preparation of endometrium-embryo cocultures during IVF.

According to a fourth aspect, the invention relates to a ready-to-use autologous endometrial coculture system intended for use for endometrium-embryo coculture during IVF, wherein said system comprises:
a culture of epithelial and stromal cells from the purified third endometrial fraction;
a semisolid IVF culture medium, for the aforementioned culture;
a sterile packaging for the culture system comprising the aforementioned culture and the aforementioned culture medium.

The invention now will be described in detail.

The present invention has as an object, according to a first aspect, a method for the preparation of a purified mixed endometrial fraction comprising stromal and glandular cells from an endometrial biopsy. The biopsy is taken during the luteal phase of the patient's menstrual cycle. The biopsy is immediately placed in a transfer-specific culture medium and then maintained at a temperature between 4° C. and 8° C. throughout the transfer procedure. Transfer between the biopsy facility and the laboratory performing the cell culture must take place as rapidly as possible; in general, the biopsy is delivered to the laboratory the day after it is taken.

The endometrium consists of stromal tissue comprising mesenchymatous (stromal) cells and secretory glandular cells comprised of epithelial cells with a lumen in the center wherefrom secretion products are released.

Treatment of the biopsy according to the invention comprises two steps of lysis of the biopsy followed by a sequence of steps of separation and purification of the two cell types. “Lysis of the endometrial biopsy” means the dissolution of the tissue structure of the endometrium, thus yielding isolated cells (such as stromal cells) or cell clusters (such as secretory glandular cells).

Lysis of the biopsy can be carried out by enzymatic digestion, for example using collagenase. In another embodiment, the biopsy can be lysed mechanically.

The principle of the purification of glandular and stromal cells is based on the difference in size and weight between the small, light stromal cells in comparison with the larger, heavier glandular cells.

Once received, the biopsy is rinsed and a microbiological analysis of the transfer medium is performed. The biopsy is then cut into small pieces and the endometrial contaminants (mucus, blood, etc.) are eliminated. Then, the biopsy is either cryopreserved or treated immediately to isolate the various cell fractions.

The biopsy pieces are subjected to an initial lysis. In one embodiment, the biopsy is digested using an enzyme, such as collagenase.

The lysate is homogenized and then filtered, thus yielding a filtrate F1 and a deposit D1 rich in glandular cells (which corresponds to the first endometrial fraction).

The aforementioned filtrate F1 is centrifuged for approximately 5 min at approximately 400 g, yielding a pellet P1.

The lysis step is repeated at least once, as follows: the deposit D1 rich in glandular cells is taken up in a suitable medium to yield a suspension S1 comprising, in addition to glandular cells, yet undigested pieces of endometrium. “Suitable medium” means any solution assuring the viability and the optimal functionality of a given population of cells.

The suspension S1 is subjected to a second lysis, thus yielding a second lysate. The latter is homogenized and then filtered, thus yielding a filtrate F2 and a deposit D2 rich in glandular cells.

If the endometrial biopsy is highly compact, a third lysis of the deposit D2 may be necessary.

The filtrate F2 is centrifuged for approximately 5 min at approximately 400 g, yielding a pellet P2.

The pellet P1 is taken up in a suitable medium, thus yielding a suspension S2 which will also be used to take up the pellet P2. A suspension S3, rich in roughly isolated stromal cells and corresponding to the second endometrial fraction, is thus obtained.

Said suspension S3 is left to sediment for approximately 30-60 min, thus yielding a supernatant SN1 rich in stromal cells and a pellet P3 comprising a third endometrial fraction, referred to as the mixed fraction, which comprises a mixture of stromal and glandular cells.

In a characteristic way for the invention, it is possible to separate the glandular and stromal cells from said third endometrial fraction in order to count them to better understand and control the culture.

a) Obtaining Stromal Cells from the Third Endometrial Fraction

The pellet P3 is taken up in a suitable culture medium to yield a suspension S4 (step al) which is then filtered, thus yielding a filtrate and a fraction retained on the filter FR1 (step a2).

The filtrate is centrifuged for approximately 5 min at 400 g, which yields a pellet P4 containing stromal cells from the third endometrial fraction (step a3). The pellet P4 is taken up in an IVF medium, thus yielding a suspension S5 of stromal cells from the third endometrial fraction (step a4). Viability studies and cell counts are then carried out on said suspension S5.

“IVF medium” means any culture medium used by procreation laboratories for procedures involving gametes and embryos.

b) Obtaining Glandular Cells from the Third Endometrial Fraction

In addition, the fraction retained FR1 is filtered, making it possible to recover glandular cells. The fraction retained FR2 thus obtained is then placed in suspension, thus yielding a suspension S6 containing glandular cells from the third endometrial fraction, in an IVF medium. A cell count of purified glandular cells is carried out on the suspension S6.

c) Mixing Glandular and Stromal Cells from the Purified Third Endometrial Fraction

The suspensions S5 and S6 are then mixed to yield a purified third endometrial fraction (step c).

The mixture of glandular and stromal cells from the purified third endometrial fraction obtained in step c) is then placed in culture in an IVF medium. The epithelial cells (arising from glands) and the stromal cells are left in culture for several days.

In a preferred embodiment, the endometrial biopsy is cryopreserved immediately, in which case, approximately on the day the patient begins ovulating, the biopsy is thawed and treated according to the method described, and then the glandular and stromal cells from the purified third endometrial fraction are placed in culture for several days.

In another embodiment, the endometrial biopsy is treated according to the method of the invention immediately after it is received by the laboratory. The mixture of glandular and stromal cells from the purified third endometrial fraction thus obtained is placed in culture for several days; said cellular culture is cryopreserved and thawed approximately on the day the patient's oocytes are retrieved.

In all cases, the method for the preparation of an autologous endometrial coculture system intended for use for endometrium-embryo coculture during IVF may comprise an additional step of packaging of the aforementioned autologous endometrial coculture system by means of a semisolid or liquid IVF medium, for use during transfer to a medically assisted procreation laboratory.

The autologous endometrial coculture system packaging comprises the preliminary preparation of a semisolid IVF medium using a concentrated agarose solution. The agarose solution and the IVF medium are mixed together. The resulting mixture is then distributed uniformly in sterile plates and stored at 4° C.

On the day of in vitro fertilization, the autologous endometrial cell culture must be ready to be sent to the medically assisted procreation laboratory in order to receive the embryo. The requirement is to have a cell layer with a minimum of 60% confluence.

In one embodiment, the semisolid IVF medium, equilibrated in advance at 37° C. in an atmosphere containing 5% CO₂ for at least 2 hours, is deposited on the cell layer. The plate is then placed in a sterile blister pack and the whole is sealed with a sterile cover. The air inside the blister pack contains 5% CO₂. The blister pack is packaged within a second, larger blister pack. The packaging thus obtained is a double blister pack.

In another embodiment, the wells containing the cell layer are filled with 500 μl/well of liquid culture medium (equilibrated in advance at 37° C. in an atmosphere containing 5% CO₂ for at least 2 hours) and then the wells are sealed with silicone caps. The plate is then placed in a sterile blister pack and the whole is sealed with a sterile cover. The blister pack is packaged within a second, larger blister pack. The packaging thus obtained is a double blister pack.

Transfer to the medically assisted procreation laboratory is carried at approximately 27° C., within 36 hours, with as little disturbance to the packaging as possible. Isothermal packaging is placed inside a carton suitable for transporting extremely fragile items. One or two eutectic gel packs are placed within the aforementioned isothermal packaging, thus allowing the temperature to be maintained at approximately 27° C. The blister pack containing the autologous endometrial cell culture is then placed on the gel pack or between the gel packs.

According to a second aspect, the invention relates to an autologous endometrial coculture system intended for use for endometrium-embryo coculture during IVF, the aforementioned system comprising a culture of epithelial and stromal cells from the purified third endometrial fraction.

In the known endometrial biopsy treatment methods, it is the epithelial cells, obtained by placing in culture glandular cells arising from the process of purifying the deposit D2 (first endometrial fraction), which are regarded as being the most desirable. Consequently, said epithelial cells are used for endometrium-embryo coculture, optionally in a mixture with purified stromal cells from the supernatant SN3 (second endometrial fraction). In all the known methods, the third endometrial fraction, referred to as the mixed fraction, contained in the pellet P3 is eliminated.

However, the applicant has noted that the culture of epithelial and stromal cells from the purified mixed endometrial fraction is of higher quality than that of purified epithelial cells from the deposit D2.

Moreover, the culture of epithelial and stromal cells from the purified mixed endometrial fraction is easily produced, which is not the case for the culture of epithelial cells arising from the first endometrial fraction.

According to a third aspect, the invention relates to the use of one such autologous endometrial coculture system as described for the preparation of endometrium-embryo cocultures during IVF.

According to a fourth aspect, the invention relates to a ready-to-use autologous endometrial coculture system intended for use for endometrium-embryo coculture during IVF, wherein said system comprises:
a culture of epithelial and stromal cells from the purified third endometrial fraction;
a semisolid IVF culture medium, for the aforementioned culture;
a sterile packaging for the culture system comprising the aforementioned culture and the aforementioned culture medium.

1. A method for the preparation, from an endometrial biopsy, of an autologous endometrial coculture system intended for use for endometrium-embryo coculture during IVF, the aforementioned culture system comprising epithelial cells and stromal cells, wherein the aforementioned method comprises steps yielding a first endometrial fraction referred to as the epithelial fraction, a second endometrial fraction referred to as the stromal fraction and a third endometrial fraction referred to as the mixed fraction which comprises a mixture of glandular and stromal cells and wherein said method comprises in addition a step of purification of the aforementioned third fraction.

2. A method according to claim 1, wherein the step of purification of the aforementioned third fraction comprises: a) obtaining stromal cells from the third fraction, b) obtaining glandular cells from the third fraction and c) mixing the stromal and glandular cells thus obtained.

3. A method according to claim 2, wherein the step a) comprises the operations of:
a1. placing in suspension the aforementioned third endometrial fraction in a suitable culture medium, thus yielding a suspension S4;
a2. filtration of the suspension S4, thus yielding a filtrate and a fraction retained on the filter FR1;
a3. centrifugation of the aforementioned filtrate, thus yielding a pellet P4 containing stromal cells from the third endometrial fraction;
a4. taking up the pellet P4 in an IVF medium, thus yielding a suspension S5 of stromal cells from the third endometrial fraction.

4. A method according to one of the claims 2 or 3, wherein the step b) comprises filtration of the aforementioned first fraction retained FR1 and placing in suspension of the fraction retained FR2 thus obtained, thus yielding a suspension S6 containing glandular cells from the third endometrial fraction, in an IVF medium.

5. A method according to claim 4, wherein the step c) consists of mixing the suspensions S5 and S6 to yield a purified third endometrial fraction.

6. A method according to claim 5, said method comprising in addition a step of placing in culture glandular and stromal cells from the purified third endometrial fraction.

7. A method according to any of the claims 1 to 3, said method comprising an additional step of packaging the aforementioned autologous endometrial coculture system by means of a semisolid or liquid IVF medium, for use during transfer to a medically assisted procreation laboratory.

8. A method according to claim 6, wherein the biopsy is treated immediately after it is received by the laboratory and the cell culture thus obtained is cryopreserved and then thawed approximately on the day the patient's oocytes are retrieved.

9. A method according to any of the claims 1 to 3, wherein the endometrial biopsy is cryopreserved immediately and the glandular and stromal cells from the purified third endometrial fraction are placed in culture approximately on the day the patient's oocytes are retrieved, after thawing and treatment of the aforementioned biopsy.

10. A method according to any of the claims 1 to 3, said method comprising at least one step of lysis of the endometrial biopsy.

11. A method according to claim 10, wherein said lysis is carried out with collagenase.

12. A method according to claim 10, wherein said lysis is carried out mechanically.

13. An autologous endometrial coculture system intended for use for endometrium-embryo coculture during IVF, wherein said method comprises epithelial and stromal cells from the purified third endometrial fraction.

14. Use of the autologous endometrial coculture system obtained by the implementation of the method according to any of the claims 1 to 3 for the preparation of endometrium-embryo cocultures during IVF.

15. A ready-to-use autologous endometrial coculture system intended for use for endometrium-embryo coculture during IVF, wherein said system comprises:
a culture of epithelial and stromal cells from the purified third endometrial fraction;
a semisolid or liquid IVF culture medium, for the aforementioned culture;
a sterile packaging for the culture system comprising the aforementioned culture and the aforementioned culture medium.
